(19) United States
(12) Patent Application Publication
(10) Pub. No.: US 2020/0263208 A1
(43) Pub. Date: Aug. 20, 2020

(54) ETHANOL PRODUCTION METHOD AND ETHANOL FERMENTATION LIQUID

(71) Applicant: Toray Industries, Inc., Tokyo (JP)

(72) Inventors: Hiroyuki MIZUNO, Kamakura (JP); Takashi MIMITSUKA, Kamakura (JP); Masashi HIGASA, Kamakura (JP); Shinjiro SAWADA, Kamakura (JP)

(21) Appl. No.: 16/645,192

(22) PCT Filed: Sep. 14, 2018
(86) PCT No.: PCT/JP2018/034116

§ 371 Date: Mar. 6, 2020

(30) Foreign Application Priority Data
Sep. 15, 2017 (JP) 2017-177482

(51) Int. Cl. C12P 7/08; C12N 1/16

(52) U.S. Cl. CPC C12P 7/08; C12N 1/16

(57) ABSTRACT

A method of producing ethanol includes: a continuous ethanol fermentation step including culturing a microorganism with a fermentation feedstock containing cane molasses as a main component, filtering the resulting culture liquid through a separation membrane to recover a filtrate containing the ethanol and from which the microorganism has been removed; retaining or returning an unfiltered liquid containing the microorganism, in or to the culture liquid; and adding an additional fermentation feedstock to the culture liquid, and an ethanol concentration and purification step including distilling the filtrate collected in the continuous ethanol fermentation step and contains the ethanol, wherein the microorganism causes a centrifugal supernatant of the culture liquid to contain particles having an average particle diameter of 100 nm or more, and particles formed by the microorganism culture and contained in the filtrate containing ethanol have an average particle diameter of 40 to 80 nm.

### TECHNICAL FIELD

This disclosure relates to a method of producing ethanol with a fermentation feedstock containing cane molasses as a main component and to an ethanol fermentation liquid.

### BACKGROUND

Production of alcohol by fermentation is an old field of study and, against a background of rising awareness of the global environment in the whole world, soaring petroleum prices and the like, particularly a technology of producing bio-ethanol by fermentation has again been attracting attention in recent years as a technology that makes it possible to suppress consumption of petroleum resources, decrease the amount of emissions of carbon dioxide, and produce sustainable fuels and industrial feedstocks.

Ethanol is generally obtained as a culture product produced by microorganisms using, as a raw material, glucose which is a hexose purified from edible biomass such as maize, or cane molasses generated in the process of purifying sugar from sugar cane. Cane molasses is consumed in large quantities as an ethanol fermentation feedstock and serves as an important fermentation feedstock in sugar-producing countries such as Brazil, Thailand and the like.

Examples of common methods of producing ethanol by microorganism culture include batch fermentation, fed-batch fermentation, continuous fermentation and the like, and WO 2007/097260 discloses that the production rate and yield of ethanol are enhanced by continuous fermentation carried out using a separation membrane. However, WO 2007/097260 includes no description of the use of a cane-molasses-containing feedstock. In addition, WO 2011/135588 discloses a method in which a culture liquid obtained by a continuous fermentation method using linked fermenters is centrifuged to separate the culture liquid into microorganisms and an ethanol fermentation liquid, the ethanol fermentation liquid from which the microorganisms have been removed is distilled, and the microorganisms are returned to the fermenters. According to WO 2011/135588, however, no separation membrane is used although cane-molasses-containing feedstock is used. It is understood that the thus obtained ethanol fermentation liquid is subsequently distilled to concentrate and purify the ethanol.

Industrial distillation is classified into batch distillation and continuous distillation. Ethanol for fuel is a chemical product for mass consumption and, thus, needs to be treated in large quantities, in which instance, continuous distillation is generally carried out.

With that ethanol fermentation liquid distillation, there is a problem in that if foaming phenomenon occurs in a distillation column, the pressure loss is increased, and flooding is finally caused to make it difficult to continue the operation of continuous distillation. A general solution to this problem is to add an antifoaming agent, but that solution costs a lot and, in addition, the antifoaming agent itself mixes, as foreign matter, into the overhead liquid from the top of the distillation column or the bottom liquid from the bottom of the distillation column. Furthermore, the antifoaming agent remaining in the distillation column adversely affects the distillation and, thus, adding an antifoaming agent is considered to be an undesirable means.

In view of this, JP 06-335627 A discloses a method in which, to suppress foaming during distillation, stirring blades attached to a stirrer shaft in the bottom of the distillation column are rotated to prevent foaming However, such a distillation column requires regular washing off of the dirt stuck during operation and, thus, moving portions and complicated structure are undesirable for such a column. Because of this, there is still a situation where adding an antifoaming agent is relied on to suppress foaming

In the same manner as described in WO 2011/135588, an ethanol fermentation method was carried out using a fermentation feedstock containing cane molasses as a main component and the genus Schizosaccharomyces as a microorganism, and the distillation was studied. As a result, it was confirmed that, as conventionally known, such a distillation process heavily generates foam and requires addition of an antifoaming agent.

It could therefore be helpful to provide a method that allows distillation to be carried out without adding an antifoaming agent during the distillation and provide such an ethanol fermentation liquid.

### SUMMARY

We discovered that an ethanol fermentation liquid obtained by a continuous fermentation method using a separation membrane surprisingly generates no foam at all during distillation even though the ethanol fermentation liquid is one which is produced using a fermentation feedstock containing cane molasses as a main component. We thus provide (1) to (8):

(1) A method of producing ethanol, including:

a continuous ethanol fermentation step including:

culturing a microorganism with a fermentation feedstock containing cane molasses as a main component;

filtering the resulting culture liquid through a separation membrane to recover a filtrate which contains the ethanol and from which the microorganism has been removed;

retaining or returning an unfiltered liquid containing the microorganism, in or to the culture liquid; and

adding an additional fermentation feedstock to the culture liquid; and

an ethanol concentration and purification step including distilling the filtrate which is collected in the continuous ethanol fermentation step and contains the ethanol;

wherein the microorganism causes a centrifugal supernatant of the culture liquid to contain particles having an average particle diameter of 100 nm or more, and

wherein particles which are formed by the microorganism culture and contained in the filtrate containing ethanol have an average particle diameter of 40 to 80 nm.

(2) The method of producing ethanol according to (1), wherein the particles have an average particle diameter of 300 nm or more.

(3) The method of producing ethanol according to (1) or (2), wherein the microorganism is a yeast belonging to the genus Schizosaccharomyces.

(4) The method of producing ethanol according to any one of (1) to (3), wherein the particles which are formed by the microorganism culture and contained in the filtrate containing ethanol have a particle diameter distribution in a particle diameter range of from 20 to 100 nm.

(5) The method of producing ethanol according to any one of (1) to (4), wherein the distillation is continuous distillation.

(6) An ethanol fermentation liquid, comprising particles which are other than the microorganism produced by the microorganism culture and have an average particle diameter of 40 to 80 nm, wherein the ethanol fermentation liquid does not contain a component generated from hydrothermally-processed bagasse.

(7) The ethanol fermentation liquid according to (6), wherein the particles have a particle diameter distribution in a particle diameter range of from 20 to 100 nm.

(8) An ethanol fermentation liquid, which shows a transmission of more than 91% T when irradiated with a beam having a wavelength of 600 nm, when the ethanol fermentation liquid is diluted to show a transmission of 0.5±0.1% T when irradiated with a beam having a wavelength of 300 nm.

By using the ethanol-containing filtrate recovered in the continuous ethanol fermentation step or the ethanol fermentation liquid in the distillation step, the foaming during distillation can be markedly suppressed and stable production of bio-ethanol through distillation can be attained, even though the ethanol fermentation liquid is one which is produced using fermentation feedstock containing, as a main component, cane molasses which is foamable during distillation.

### BRIEF DESCRIPTION OF THE DRAWINGS

FIG. 1 shows the results of particle diameter distribution of ethanol fermentation liquids 1 to 3.

FIG. 2 shows the result of particle diameter distribution of the ethanol fermentation liquid 1 (enlarged).

### DETAILED DESCRIPTION

Our method includes a continuous ethanol fermentation step using a separation membrane with a microorganism cultured with a fermentation feedstock containing cane molasses as a main component, wherein the microorganism causes a centrifugal supernatant of the resulting culture liquid to contain particles having an average particle diameter of 100 nm or more, and a concentration and purification step including distilling the ethanol filtrate recovered in the continuous fermentation step; and relates to an ethanol fermentation liquid containing particles which are formed by the microorganism culture and have a specific average particle diameter. Below, a method of producing ethanol will be described step by step and, in addition, the characteristics of the ethanol fermentation liquid will be described.

### Continuous Ethanol Fermentation Step

The microorganism is a microorganism having the capability to produce ethanol and, without particular limitation, may be any microorganism which causes a centrifugal supernatant of a culture liquid to contain particles having an average particle diameter of 100 nm or more, wherein the culture liquid is obtained by culturing the microorganism with a fermentation feedstock containing cane molasses as a main component. Preferable specific examples of such microorganisms include: yeasts such as baker's yeast frequently used in the fermentation industry; bacteria such as E. Coli and coryneform bacteria; filamentous fungi; Actinomyces; and the like. Such microorganisms may be isolated from the natural environment, or may also be ones the nature of which is partially altered by mutation or genetic recombination. A microorganism used to produce ethanol is preferably yeast. Preferable examples of yeasts include the genus Saccharomyces, the genus Kluyveromyces, and the genus Schizosaccharomyces. Among these, yeasts belonging to the genus Schizosaccharomyces are preferable, and Schizosaccharomyces pombe, Schizosaccharomyces japonicus, Schizosaccharomyces octosporus, or Schizosaccharomyces cryophilus can be suitably used.

The particle refers to an insoluble particulate substance contained in a culture liquid obtained by culturing a microorganism with a fermentation feedstock containing cane molasses as a main component, and is other than the microorganism. The average particle diameter of the particles present in the culture liquid is measured by dynamic light scattering (DLS, a photon correlation method). Specifically, an autocorrelation function is determined by cumulant analysis from a fluctuation in a scattering intensity obtained by dynamic light scattering measurement. The autocorrelation function is converted to a particle size distribution relative to the scattering intensity and then, the conversion result is converted to an average particle diameter in an analysis range of from the minimum value of 1 nm to the maximum value of 5000 nm. For the measurement, the ELS-Z2 manufactured by Otsuka Electronics Co., Ltd. is used. In addition, because the microorganism is also present as particles in the culture liquid, the culture liquid at room temperature is centrifuged under the conditions at 1,000 G for 10 minutes to precipitate the microorganism, and the average particle diameter of the particles contained in the centrifugal supernatant is measured.

The particles contained in the culture liquid have an average particle diameter of 100 nm or more, preferably 300 nm or more, more preferably 300 to 1500 nm. As shown in the below-mentioned Examples, use of a microorganism that causes a culture liquid to contain such particles having an average particle diameter of 100 nm or more brings about the unexpected excellent effect of suppressing the foaming of an ethanol-containing filtrate recovered in the continuous ethanol fermentation step and is used for distillation, although no detailed action mechanism is clear. In this regard, the upper limit of the average particle diameter of the particles is not limited to any particular value to the extent that the filtration flux is not reduced by the occurrence of membrane clogging, but the upper limit is the average particle diameter of such particles which are not precipitated together with a microorganism through the centrifugation, and the preferable upper limit value is 1500 nm.

Cane molasses is a byproduct produced in the process of sugar production from sugar cane squeezed juice or raw sugar. Specifically, cane molasses refers to a crystallization mother liquor containing a sugar component remaining after crystallization in a crystallization step in a sugar production process. In general, the crystallization step is carried out usually a plurality of times, in which crystallization is repeated such that a first crystallization is carried out to obtain a crystal component as a first sugar, a further crystallization of the residual liquid (a first molasses) from the first sugar is carried out to obtain a crystal component as a second sugar, a still further crystallization of the residual liquid (a second molasses) from the second sugar is carried out to obtain a third sugar, and so on, and the molasses obtained at the final stage as a crystallization mother liquor remaining from the step is called cane molasses. As the number of times of crystallization increases, inorganic salts other than sugar components are more concentrated in cane molasses. As the cane molasses, cane molasses that has undergone crystallization many times is preferable, and preferable cane molasses results from crystallization carried out at least two times or more, more preferably three times or more. The sugar components contained in cane molasses include sucrose, glucose, and fructose as main components, and may include other sugar components in slight amounts such as xylose and galactose. The sugar concentration of cane molasses is generally about 200 to 800 g/L. The sugar concentration of cane molasses can be quantified by a known measurement technique such as HPLC.

A fermentation feedstock means one containing all nutrients required for the growth of microorganisms. The fermentation feedstock only needs to contain cane molasses as a main component and, in addition, a carbon source(s), nitrogen source(s), inorganic salt(s), and, if necessary, organic micronutrient(s) such as amino acid(s) and vitamin(s) may be suitably added. In this regard, a fermentation feedstock containing cane molasses as a main component means that 50 weight percent or more of the matter (not including water) contained in the fermentation feedstock is cane molasses.

Examples of carbon sources to be preferably used include: saccharides such as glucose, sucrose, fructose, galactose, and lactose; corn starch saccharified liquids containing these saccharides; sweet potato molasses, sugar beet molasses, and high test molasses; organic acids such as acetic acid; alcohols such as ethanol; glycerin; and besides, sugar liquids derived from cellulose-containing biomass.

Examples of cellulose-containing biomass include: plants-based biomass such as bagasse, switchgrass, corn stover, rice straw, and wheat straw; wood-based biomass such as trees and waste construction materials and the like. Cellulose-containing biomass contains cellulose or hemicellulose which is a polysaccharide resulting from dehydration condensation of sugar, and hydrolysis of such a polysaccharide allows production of a sugar liquid usable as a fermentation feedstock.

A method of preparing a sugar liquid derived from cellulose-containing biomass is not limited to any particular one, and examples of disclosed methods of producing such a sugar include: a method in which a sugar liquid is produced by acid hydrolysis of biomass using a concentrated sulfuric acid (JP H11-506934 W, JP 2005-229821 A); and a method in which a sugar liquid is produced by hydrolysis treatment of biomass using a diluted sulfuric acid and then further by enzymatic treatment using cellulase or the like (A. Aden, “Lignocellulosic Biomass to Ethanol Process Design and Economics Utilizing Co-Current Dilute Acid Prehydrolysis and Enzymatic Hydrolysis for Corn Stover”, NREL Technical Report (2002)). In addition, examples of disclosed methods in which no acid is used include: a method in which a sugar liquid is produced by hydrolysis of biomass using subcritical water at about 250 to 500° C. (JP 2003-212888 A); a method in which a sugar liquid is produced by subcritical water treatment of biomass and then further by enzymatic treatment of the biomass (JP 2001-95597 A); and a method in which a sugar liquid is produced by hydrolysis treatment of biomass using hot water at about 240 to 280° C. under pressure and then further by enzymatic treatment of the biomass (JP 3041380 B). After the above-mentioned treatments, the obtained sugar liquid and cane molasses may be mixed and purified. Such a method is disclosed in, for example, WO 2012/118171.

Examples of nitrogen sources to be used include: ammonia gas, aqueous ammonia, ammonium salts, urea, and nitric acid salts; other organic nitrogen sources to be supplementarily used, for example, oil cakes, soy bean hydrolysate liquids, casein degradation products, other amino acids, vitamins, corn steep liquors, yeasts or yeast extracts, meat extracts, peptides such as peptone, and various fermentation microorganisms and hydrolysates thereof and the like.

As an inorganic salt, phosphate, magnesium salt, calcium salt, iron salt, manganese salt or the like can be suitably added, if necessary.

In addition, when a microorganism requires a specific nutrient in order to grow, the nutritive substance can be added as a purified product or a natural product containing the substance.

The above-mentioned continuous ethanol fermentation step carried out using the microorganisms and the fermentation feedstock is a continuous fermentation step carried out using a separation membrane, and is specifically a continuous fermentation step characterized in that a culture liquid is filtered through a separation membrane to recover an ethanol-containing filtrate from which the microorganisms have been removed, that an unfiltered liquid containing microorganisms is retained in or returned to the culture liquid, and that an additional fermentation feedstock is added to the culture liquid.

The separation membrane used in the continuous ethanol fermentation step is not limited to any particular one and may be any of those which have the function of separating, from microorganisms by filtration, the fermentation liquid obtained by microorganism culture, and examples of usable materials include porous ceramic membranes, porous glass membranes, porous organic polymer membranes, metallic fiber textiles, nonwoven fabrics and the like, among which particularly porous organic polymer membranes or ceramic membranes are preferred.

From the viewpoint of resistance to dirt, the separation membrane is preferably structured, for example, as a separation membrane containing a porous resin layer as a functional layer.

The separation membrane having a porous resin layer preferably has, on the surface of a porous base material, a porous resin layer that acts as a separation function layer. The porous base material supports the porous resin layer to give strength to the separation membrane. When the separation membrane has a porous resin layer on the surface of a porous base material, the porous base material may be impregnated with the porous resin layer, or may not be impregnated with the porous resin layer.

The average thickness of the porous base material is preferably 50 to 3000 μm.

The porous base material is composed of an organic material and/or inorganic material and/or the like, and an organic fiber is preferably used. Examples of preferred porous base materials include woven fabrics and nonwoven fabrics composed of organic fibers such as cellulose fibers, cellulose triacetate fibers, polyester fibers, polypropylene fibers, and polyethylene fibers, and more preferably, nonwoven fabrics are used because their density can be relatively easily controlled, they can be simply produced, and they are inexpensive.

As the porous resin layer, an organic polymer membrane can be preferably used. Examples of organic polymer membrane materials include polyethylene resins, polypropylene resins, polyvinyl chloride resins, polyvinylidene fluoride resins, polysulfone resins, polyethersulfone resins, polyacrylonitrile resins, cellulose resins, cellulose triacetate resins and the like. The organic polymer membrane may be a resin mixture containing such a resin as a main component. The main component means that the component is contained in an amount of 50 wt % or more, preferably 60 wt % or more. Examples of preferred organic polymer membrane materials include those which can be easily formed into a membrane using a solution and have excellent physical durability and chemical resistance such as polyvinyl chloride resins, polyvinylidene fluoride resins, polysulfone resins, polyethersulfone resins and polyacrylonitrile resins, and polyvinylidene fluoride resins or resins containing them as a main component are most preferably used.

As the polyvinylidene fluoride resin, a homopolymer of vinylidene fluoride is preferably used. Further, as the polyvinylidene fluoride resin, a copolymer of vinylidene fluoride and a vinyl monomer capable of copolymerizing therewith is also preferably used. Examples of vinyl monomers capable of copolymerizing with vinylidene fluoride include tetrafluoroethylene, hexafluoropropylene, ethylene fluoride trichloride and the like.

The separation membrane has only to have a pore size that does not allow the passage of the microorganism used in the culture, and the pore size is desirably in a range such that the separation membrane is less likely to suffer clogging due to secretions of the microorganism used in the culture and fine particles in the fermentation feedstock, and stably maintains its filtration performance for a long time. Thus, the average pore size of the porous separation membrane is preferably 0.01 to 5 μm. More preferably, when the average pore size of the separation membrane is 0.01 to 1 μm, both a high rejection rate which does not allow leakage of the microorganism and high water permeability can be achieved, and the water permeability can be retained for a long time.

The average pore size of the separation membrane is preferably 1 μm or less because, when the average pore size is close to the size of the microorganism, the pores may be directly clogged with the microorganism. From the viewpoint of preventing leakage of the microorganism, that is, preventing the occurrence of a trouble causing a decrease in the rejection rate, the average pore size of the separation membrane is preferably not too large relative to the size of the microorganism. When bacteria whose cells are small or the like are used as the microorganism, the average pore size is preferably 0.4 μm or less, more preferably 0.2 μm or less, still more preferably 0.1 μm or less. Too small an average pore size reduces the water permeability of the separation membrane, which then does not enable efficient operation even though the separation membrane is not fouled and, accordingly, the average pore size of the separation membrane is preferably 0.01 μm or more, more preferably 0.02 μm or more, still more preferably 0.04 μm or more.

The average pore size can be determined by measuring the diameters of all pores which can be observed within an area of 9.2 μm×10.4 μm under a scanning electron microscope at a magnification of 10,000×, and averaging the measured values. Alternatively, the average pore size can be determined by: taking a picture of the surface of a membrane using a scanning electron microscope at a magnification of 10,000×; randomly selecting 10 or more pores, preferably 20 or more pores; measuring the diameters of these pores; and calculating the number average. When the pore is not circular, a circle having the same area as the pore has (equivalent circle) can be determined using an image processing device or the like, and the diameter of the equivalent circle is regarded as the diameter of the pore.

The standard deviation σ of the average pore size of the separation membrane is preferably 0.1 μm or less. The smaller the standard deviation 6 of the average pore size, the better. The standard deviation σ of the average pore size is calculated according to Equation (1), wherein N represents the number of pores observable within the above-mentioned area of 9.2 μm×10.4 μm; Xₖ represents the respective measured diameters; and X(ave) represents the average of the pore sizes.

$$σ = \sqrt{\frac{{\sum }_{k = 1}^{N} {( {X}_{k} - X ( a v e ) )}^{2}}{N}} \qquad ( 1 )$$

In the separation membrane, its permeability for a culture liquid is one of its important properties. As an index of the permeability of the separation membrane, the pure water permeability coefficient of the separation membrane before use can be used. The pure water permeability coefficient of the separation membrane is preferably 5.6×10⁻¹⁰ m³/m²/s/pa or more as calculated when the amount of water permeation is measured at a head height of 1 m using purified water having a temperature of 25° C. and prepared with a reverse osmosis membrane. When the pure water permeability coefficient is 5.6×10⁻¹⁰ m³/m²/s/pa to 6×10⁻⁷ m³/m²/s/pa, a practically sufficient amount of water permeation can be obtained.

The surface roughness of the separation membrane means the average height in the direction perpendicular to the surface. The membrane surface roughness is one of the factors that enable the microorganism adhering to the surface of the separation membrane to be detached more easily by the membrane surface washing effect of a liquid current generated by stirring or a circulating pump. The surface roughness of the separation membrane is not limited to any particular value but has only to be in a range such that the microorganism and other solids adhering to the membrane can be detached, and the surface roughness is preferably 0.1 μm or less. When the surface roughness is 0.1 μm or less, the microorganism and other solids adhering to the membrane can be easily detached.

The separation membrane more preferably has a surface roughness of 0.1 μm or less, an average pore size of 0.01 to 1 μm, and a pure water permeability coefficient of 2×10⁻⁹ m³/m²/s/pa or more, and using such a separation membrane has revealed that the operation can be more easily carried out without requiring excessive power for washing the membrane surface. When the separation membrane surface roughness is 0.1 μm or less, the shear force generated on the membrane surface can be reduced during the filtration of the microorganism, destruction of the microorganism can be suppressed, and clogging of the separation membrane can be suppressed so that stable filtration can be more easily carried out for a long time. When the membrane surface roughness of the separation membrane is 0.1 μm or less, continuous fermentation can be carried out with a smaller transmembrane pressure difference, and the membrane can be more easily restored by washing than when the operation is carried out with a large transmembrane pressure difference, even if the separation membrane is clogged. Because suppression of the clogging of the separation membrane enables stable continuous fermentation, the surface roughness of the separation membrane is preferably as small as possible.

The membrane surface roughness of the separation membrane is measured using the following atomic force microscope (AFM) under the following conditions:

Apparatus: an atomic force microscope apparatus (“Nanoscope IIIa” manufactured by Digital Instruments, Inc.)
Conditions: Probe: an SiN cantilever (manufactured by Digital Instruments, Inc.)
Scanning mode: contact mode (measurement in air)
   underwater tapping mode (underwater measurement)
Scanning area: 10 μm square, 25 μm square (measurement in air)
   5 μm square, 10 μm square (underwater measurement)
Scanning resolution: 512×512
   Sample preparation: for the measurement, the membrane sample was soaked in ethanol at room temperature for 15 minutes, and then soaked in RO water for 24 hours, followed by washing and drying it in the air. The RO water means water prepared by filtration through a reverse osmosis membrane (RO membrane), which is a filtration membrane, to remove impurities such as ions and salts. The pore size of the RO membrane is about 2 nm or less.

The membrane surface roughness, drough, is calculated according to Equation (2) on the basis of the height of each point measured in the direction of the Z-axis using the above atomic force microscope apparatus (AFM).

$${d}_{rough} = {\sum }_{n = 1}^{N} \frac{\vert {Z}_{n} - \overline{Z} \vert}{N} \qquad ( 2 )$$

d_{rough}: surface roughness (μm)
Zₙ: height in the direction of Z-axis (μm)
Z̅: average height in scanning area (μm)
N: number of measurement samples

The separation membrane is not limited to any particular shape, but a flat membrane, a hollow fiber membrane or the like can be used, and a hollow fiber membrane is preferable. When the separation membrane is a hollow fiber membrane, the inner diameter of the hollow fiber is preferably 200 to 5000 μm, and the membrane thickness is preferably 20 to 2000 μm. Textile or knit produced by forming an organic fiber or an inorganic fiber into a cylindrical shape may be contained in the hollow fiber.

The above-mentioned separation membrane can be produced by, for example, the production method described in WO 2007/097260.

In the continuous ethanol fermentation step, the transmembrane pressure difference during the filtration is not limited to any particular value but is acceptable as long as the filtration of the culture liquid is possible. However, when filtration treatment is carried out using an organic polymer membrane with a transmembrane pressure difference of more than 150 kPa to filter a culture liquid, the structure of the organic polymer membrane is more likely to be destroyed, and this may lead to the lowered capability to produce ethanol. In addition, with a transmembrane pressure difference of less than 0.1 kPa, the amount of water permeation of the culture liquid is often insufficient, and thus, the productivity in production of ethanol tends to be low. Thus, in the method of producing ethanol, a transmembrane pressure difference of 0.1 to 150 kPa as the filtration pressure is preferably used for an organic polymer membrane, whereby the amount of permeation of the culture liquid is large, and there is no decrease in the capacity to produce the ethanol due to destruction of the membrane structure so that the capability to produce the ethanol can be kept high. For organic polymer membranes, the transmembrane pressure difference is preferably 0.1 to 50 kPa, more preferably 0.1 to 20 kPa.

The temperature during the culture by a yeast can be set to a temperature suitable for the yeast used, and is not limited to any particular value as long as it is within a range in which the microorganism can grow, and the fermentation is carried out in a temperature range of 20 to 75° C.

In the method of producing ethanol, batch fermentation or fed-batch fermentation may be carried out in the initial phase of the culture to increase the microorganism concentration and, after this, continuous fermentation (filtration of the fermentation liquid) may be started. Alternatively, a high concentration of microorganisms may be seeded, and continuous fermentation may be started upon start of culture. In a method of producing ethanol, it is possible to start supply of the fermentation feedstock and filtration of the culture liquid at appropriate timings. The times to start the supply of the fermentation feedstock and the filtration of the culture liquid do not necessarily need to be the same. In addition, the supply of the fermentation feedstock and the filtration of the culture liquid may be carried out either continuously or intermittently.

The microorganism concentration of the culture liquid is a concentration preferred to achieve efficient productivity so that the productivity of the ethanol can be maintained at a high level. A good production efficiency can be obtained by maintaining the microorganism concentration of the culture liquid at, for example, 5 g/L or more in terms of dry weight.

In the continuous ethanol fermentation step, a part of the culture liquid containing the microorganism may be removed from the fermenter, if necessary, during the continuous fermentation, and the culture liquid may then be supplied with fermentation feedstock to attain dilution to thereby control the concentration of the microorganism in the fermenter. For example, if the concentration of the microorganism in the fermenter is too high, clogging of the separation membrane is likely to occur and, in view of this, the clogging may be prevented by removing a part of the culture liquid containing the microorganism and diluting the culture liquid with the fermentation feedstock supplied. The number of fermenters is not restricted.

A continuous fermentation device is not limited to any particular one as long as it is an ethanol production device based on a continuous fermentation process including filtering a culture liquid containing a microorganism through a separation membrane and recovering ethanol from the filtrate, while retaining or returning an unfiltered liquid containing a microorganism, in or to the culture liquid, and adding an additional fermentation feedstock to the culture liquid, thereby the ethanol is recovered from the filtrate. Specific examples of usable devices include the devices described in WO2007/097260 and WO2010/038613.

### Distillation Step

As the ethanol distillation method in the method of producing ethanol, batch distillation or continuous distillation, which is an ethanol distillation method known to a person skilled in the art, can be applied, and continuous distillation is preferably applied. In a continuous distillation method, the ethanol filtrate gasified by a heater is first introduced continuously into the middle stage of a distillation column. An overhead liquid rich in ethanol, which is more volatile, is continuously obtained from the top of the distillation column, and a bottom liquid rich in less volatile components (impurities such as lactic acid and acetic acid) is continuously obtained from the bottom. Setting the total amount of the continuously obtained overhead liquid and bottom liquid to be the same as the amount of the continuously supplied feedstock allows the distillation column to be in a steady state.

A preferably usable form of the distillation column is a rectifying column having high separation performance The rectifying column may be either of a plate column and a packed column. An ethanol-containing filtrate recovered in the continuous fermentation step which is the former step is characterized by having markedly low foamability. Because of this, the continuous distillation carried out using a packed column, which is difficult to employ for a foamable liquid but requires low cost for facilities, can be preferably employed.

### Ethanol Fermentation Liquid

The ethanol-containing filtrate recovered in the continuous ethanol fermentation step contains an insoluble particulate substance (hereinafter, “particles”) which is formed by the microorganism culture, is other than the microorganism, and has an average particle diameter of 40 to 80 nm. The fact that the particles are generated by microorganism culture is itself a novel finding. Because of this, an analysis method for the composition and the like of the particles has not been established as the technical common knowledge of a person skilled in the art, and it is only clear that the particles are a byproduct formed by microorganism culture. Although no detailed action mechanism is clear, the below-mentioned Examples have found, as an unexpected excellent effect, that the particles contained in the ethanol-containing filtrate suppress the foaming of the filtrate used for distillation. Accordingly, irrespective of whether or not the particles are those obtained by the continuous ethanol fermentation step, an ethanol fermentation liquid identified by containing the particles having an average particle diameter of 40 to 80 nm is itself one aspect of this disclosure.

The average particle diameter of the particles present in the ethanol fermentation liquid is measured by dynamic light scattering (DLS, a photon correlation method). Specifically, an autocorrelation function is determined by cumulant analysis from a fluctuation in a scattering intensity obtained by dynamic light scattering measurement. The autocorrelation function is converted to a particle diameter distribution relative to the scattering intensity, and then, the conversion result is converted to an average particle diameter in an analysis range of from the minimum value of 1 nm to the maximum value of 5000 nm. For the measurement, the ELS-Z2 manufactured by Otsuka Electronics Co., Ltd. is used. In addition, the microorganism is also present as particles in the ethanol fermentation liquid in some examples, and thus, the ethanol fermentation liquid at room temperature is centrifuged under the conditions at 1,000 G for 10 minutes to precipitate the microorganism, and then the average particle diameter of the particles contained in the centrifugal supernatant is measured.

The particles contained in the ethanol fermentation liquid have an average particle diameter of 40 to 80 nm, preferably 50 to 70 nm. In addition, the particles preferably have a particle diameter distribution of 20 to 100 nm, more preferably 40 to 90 nm.

An ethanol fermentation liquid is not limited to any particular one as long as it contains the particles and may be, for example, an ethanol fermentation liquid containing the microorganisms obtained immediately after microorganism culture, may be an ethanol fermentation liquid from which the microorganisms have been removed, or may be an ethanol fermentation liquid obtained by removing the microorganisms from an ethanol fermentation liquid and suitably purifying and concentrating this liquid by a known method.

The concentration of the ethanol contained in an ethanol fermentation liquid is not limited to any particular value, and is preferably 30 to 150 g/L, more preferably 50 to 120 g/L, still more preferably 60 to 100 g/L.

In addition, although no detailed action mechanism is clear, the below-mentioned Examples have found, as an unexpected excellent effect, that an ethanol fermentation liquid further suppresses the foaming of the ethanol fermentation liquid used for distillation, when the ethanol fermentation liquid shows a transmission of more than 91% T when irradiated with a beam having a wavelength of 600 nm, as measured using the ethanol fermentation liquid diluted with water to show a transmission of 0.5±0.1% T when irradiated with a beam having a wavelength of 300 nm. Accordingly, whether or not an ethanol fermentation liquid is one obtained by the continuous ethanol fermentation step, the ethanol fermentation liquid preferably shows a transmission of more than 91% T when irradiated with a beam having a wavelength of 600 nm, more preferably 94% T or more, wherein the ethanol fermentation liquid is diluted with water to have a transmission of 0.5±0.1% T when irradiated with a beam having a wavelength of 300 nm.

The transmission of the ethanol fermentation liquid is a value measured using an ultraviolet and visible spectrophotometer. Specifically, distilled water is added to a 10 mm square quartz cuvette, and measured for transmission background when irradiated with beams having wavelengths of 200 nm to 800 nm. Then, the ethanol fermentation liquid and distilled water are mixed in an empty cuvette such that the mixture shows a transmission of 0.5 ±0.1% T when irradiated with a beam having a wavelength of 300 nm, followed by measuring a transmission which the resulting liquid shows when irradiated with a beam having a wavelength of 600 nm. As an ultraviolet and visible spectrophotometer for this measurement, an UV-Vis measurement device (V750) manufactured by JASCO Corporation can be used.

The ethanol fermentation liquid is characterized by having markedly low foamability and, thus, can preferably be used as feedstock which is used for ethanol for fuel and requires concentration and purification by distillation. Ethanol foamability can be evaluated on the basis of the volume of foam from an ethanol fermentation liquid and the height of foam from an ethanol fermentation liquid used in a test which simulates continuous distillation, as the details are explained in the below-mentioned Examples.

### EXAMPLES

Below, our methods and liquids will specifically be described with reference to Examples. However, this disclosure is not to be limited thereto.

### Reference Example 1 Method of Analyzing Saccharides and Ethanol

The concentrations of saccharides and ethanol in the feedstock were quantified under the below-mentioned HPLC conditions and on the basis of comparison with standard samples.
Column: Shodex SH1011 (manufactured by Showa Denko K. K.)
Mobile phase: 5 mM sulfuric acid (flow rate: 0.6 mL/minute)
Reaction solution: none
Detection method: RI (differential refractive index)
Temperature: 65° C.

### Reference Example 2 Preparation of Fermentation Feedstock

Cane molasses and water were mixed at a weight ratio of 1:3 to obtain a fermentation feedstock. The saccharide analysis results obtained using the method shown in Reference Example 1 are shown in Table 1.

TABLE 1

| | Glucose | Fructose | Sucrose |
|---|---|---|---|
| | (g/L) | (g/L) | (g/L) |
| Fermentation Feedstock | 15.4 | 21.6 | 79.3 |

### Example 1 Separation-Membrane-Utilized Continuous Fermentation Carried out Using Schizosaccharomyces pombe NBRC1628 Strain

Separation-membrane-utilized continuous fermentation was carried out using the Schizosaccharomyces pombe NBRC1628 strain as a microorganism and using, as a culture medium, the fermentation feedstock of Reference Example 2. A separation membrane element in the form of a hollow fiber described in JP 2010-22321 A was employed. The Schizosaccharomyces pombe NBRC1628 strain was inoculated in a test tube in which 5 ml of the fermentation feedstock of Reference Example 2 had been loaded, and the resulting liquid was subjected to shaking culture overnight (pre-preculture). The obtained culture liquid was inoculated in an Erlenmeyer flask in which 45 mL of fresh fermentation feedstock of Reference Example 2 had been loaded, and the resulting liquid was subjected to shaking culture at 30° C. at 120 rpm for eight hours (preculture). Out of 50 mL of the preculture liquid, 35 mL aliquot was taken and inoculated in a continuous fermentation device in which 700 mL of the fermentation feedstock of Reference Example 2 had been loaded, and the resulting liquid was cultured for 24 hours with stirring at 300 rpm using an accessory stirrer in a fermentation reaction vessel. In this regard, a culture liquid circulating pump was started up immediately after the inoculation to cause liquid circulation between the separation membrane element and the fermenter. Upon completion of the preculture, a filtration pump was started up to start pulling the culture liquid out of the separation membrane element. After filtration was started, fermentation feedstock was added such that the culture liquid in the continuous fermentation device could be controlled in an amount of 700 mL while continuous fermentation was carried out under the following continuous fermentation conditions for about 200 hours, whereby 700 mL of ethanol-containing filtrate (fermentation liquid sample 1) having an ethanol concentration of 64 g/L was obtained.

### Continuous Fermentation Conditions

Fermentation reaction vessel capacity: 2 (L)
Separation membrane used: filtration membrane made of polyvinylidene fluoride
Effective filter area of membrane separation element: 218 (cm²)
Temperature adjustment: 30 (° C.)
Aeration rate in fermentation reaction vessel: no aeration
Stirring rate in fermentation reaction vessel: 300 (rpm)
pH adjustment: no adjustment
Filtration flux setting value: 0.1 (m³/m²/day)
Sterilization: the separation membrane element and the fermenter were autoclaved at 121° C. for 20 minutes.
Average pore size: 0.1 μm
Standard deviation of average pore size: 0.035 μm
Membrane surface roughness: 0.06 μm
Pure water permeability coefficient: 50×10⁻⁹ m³/m²/s/pa

### Reference Example 3 Batch Fermentation Carried out Using Schizosaccharomyces pombe NBRC1628 Strain

Batch fermentation was carried out using the same fermentation feedstock, microorganism, preculture conditions, and fermentation conditions as in Example 1. However, filtering a culture liquid using a separation membrane was not carried out.

The Schizosaccharomyces pombe NBRC1628 strain was inoculated in a test tube in which 5 ml of the fermentation feedstock shown in Table 1 had been loaded, and the resulting liquid was subjected to shaking culture overnight (pre-preculture). The obtained culture liquid was inoculated in an Erlenmeyer flask in which 45 mL of fresh fermentation feedstock shown in Table 1 had been loaded, and the resulting liquid was subjected to shaking culture at 30° C. at 120 rpm for eight hours (preculture). Out of 50 mL of the preculture liquid, 35 mL aliquot was taken and inoculated in a continuous fermentation device in which 700 mL of the fermentation feedstock shown in Table 1 had been loaded, and the resulting liquid was stirred at 300 rpm using an accessory stirrer in a fermentation reaction vessel to undergo batch fermentation under the following fermentation conditions for 48 hours, whereby 700 mL of ethanol fermentation liquid (fermentation liquid sample 2) having an ethanol concentration of 58 g/L was obtained.

### Batch Fermentation Conditions

Fermentation reaction vessel capacity: 2 (L)
Temperature adjustment: 30 (° C.)
Aeration rate in fermentation reaction vessel: no aeration
Stirring rate in fermentation reaction vessel: 300 (rpm)
pH adjustment: no adjustment

### Reference Example 4 Removal of Microorganisms from Batch Fermentation Liquid

The fermentation liquid sample 2 obtained in Reference Example 3 contained microorganisms and, accordingly, the microorganisms were precipitated by centrifugation at 1,000 G for 10 minutes to obtain 600 mL of the supernatant fluid (a fermentation liquid sample 3).

### Example 2 Distillation Test of Fermentation Liquid Sample

A test was carried out, simulating the internal state of a rectifying column for continuous distillation. The fermentation liquid samples 1 to 3, 300 mL each, were separately added to 500 mL volume round bottom flasks, and each round bottom flask was operated to be heated by a mantle heater such that the liquid temperature sensor in the round bottom flask maintained 95° C. A cooling condenser was mounted at the outlet of the round bottom flask, and cooling water at 4° C. was circulated through the internal piping of the condenser to cool and condense the evaporated ethanol As a result, the fermentation liquid samples 2 and 3 so heavily foamed immediately after boiling that the foam went up to the cooling condenser. Surprisingly, the other fermentation liquid sample 1 did not foam at all even though the sample maintained a state of distillation for five hours.

### Example 3 Evaluation of Foamability

To evaluate foamability, a test was carried out using a flow-down method, which is a method of evaluating foamability using a measuring cylinder as described in Tamura, Takamitsu, “The Test Methods for Measuring Foaming and Antifoaming Properties of Liquid,” Journal of Japan Oil Chemists' Society, 42 (10): pp. 737-745, 1993. Measuring cylinders having a volume of 500 mL were set upright, the fermentation liquid samples 1 to 3, 50 mL each, were first placed separately in the measuring cylinders; the samples 1 to 3, 300 mL each, were separately allowed to flow down from the 45 cm high position; and the volume of foam generated was measured. As a result, the volume of foam from the fermentation liquid sample 1 was 0 mL, and the volumes of foam from the fermentation liquid samples 2 and 3 were 55 mL and 65 mL, respectively.

The results from Examples 2 and 3 have revealed that the fermentation liquid sample 1 is surprisingly characterized by generating no foam at all.

### Example 4 Measurement Results of Particle Diameter Distribution and Average Particle Diameter in Ethanol Fermentation Liquid

Each of the fermentation liquid samples 1 to 3 was poured into a disposable cuvette having a capacity of 1 mL and measured for average particle diameter by dynamic light scattering.

### Measurement Conditions

Pinhole size of light source: 100 μm
Measurement wavelength: 660 nm
Measurement angle: 165°
Measurement cumulated number: 70 times
Solvent refractive index: 1.3313
Solvent viscosity: 0.8852 cp

Next, the measurement results were analyzed under the following conditions.

### Analysis Conditions

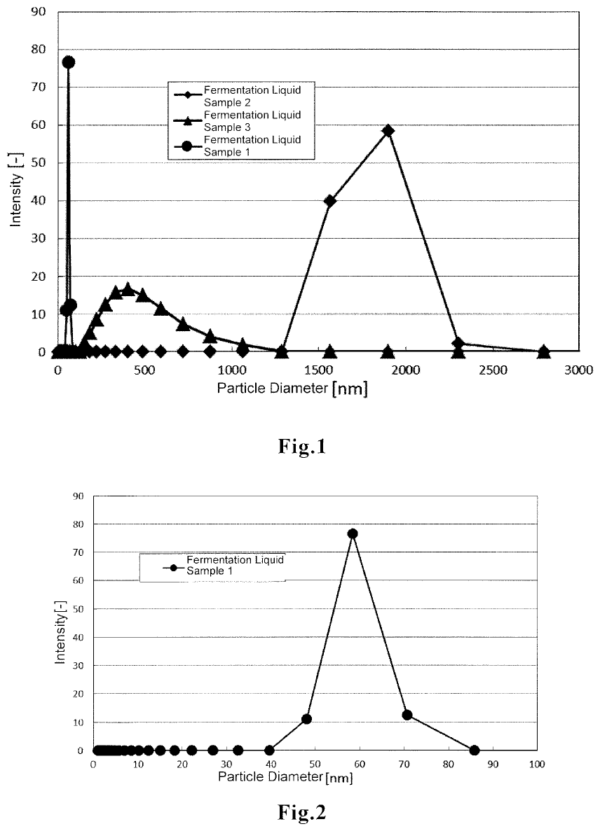

For particle diameter analysis, a zeta-potential & particle size analyzer, ELS-Z2, manufactured by Otsuka Electronics Co., Ltd. was used, and measurement was carried out in the air at 25° C. An autocorrelation function was determined by cumulant analysis from a fluctuation in a scattering intensity obtained by dynamic light scattering, and the autocorrelation function was converted to a particle size distribution relative to the scattering intensity. The histogram analysis range of the particle size distribution was from the minimum value of 1 nm to the maximum value of 5000 nm. The obtained particle diameter distribution results are shown in FIG. 1. The enlarged particle diameter distribution of only the fermentation liquid sample 1 is shown in FIG. 2. In addition, Table 2 shows the results putting together the average particle diameters and the volumes of foam of the fermentation liquid samples 1 to 3.

TABLE 2

| | Sample | | | | | |
|---|---|---|---|---|---|---|
| | | | Fermentation | | Fermentation | |
| | Fermentation | | Liquid | | Liquid | |
| | Liquid | | Sample 2 | | Sample 3 | |
| | Sample 1 | | Culture liquid | | Culture liquid | |
| | Filtrate | | obtained by batch | | obtained by removing | |
| | obtained by | | fermentation | | microorganisms from | |
| | continuous | | (containing | | Sample 2 by | |
| Features | fermentation | | microorganisms) | | centrifugation | |
| Foamability | 0 | mL | 55 | mL | 65 | mL |
| (Foam | | | | | | |
| Volume) | | | | | | |
| Average | 58 | nm | 1741 | nm | 431 | nm |
| Particle | | | | | | |
| Diameter | | | | | | |

As shown in FIG. 1 and Table 2, the results revealed that the cane molasses as feedstock contains no particles, and that particles were formed through microorganism culture. In addition, the results revealed that the ethanol fermentation liquid containing particles which are formed by microorganism culture and have an average particle diameter of 58 nm can markedly suppress foaming

### Example 5 Foamability of Ethanol Fermentation Liquid Sample

The foamability of the ethanol fermentation liquid samples was evaluated in a test which simulated continuous distillation. The fermentation liquid samples 1 to 3, 3 mL each, and stirrer chips were added to separate test tubes (transparent and scaled 20 mL common stoppered test tubes) manufactured by Tokyo Garasu Kikai Co., Ltd., and a cooling pipe set to 10° C. was mounted to the upper portion of each test tube. The test tube was placed in a temperature-controllable oil bath manufactured by Tokyo Rikakikai Co., Ltd. with the temperature set to 160° C., such that the liquid level of the fermentation liquid in the test tube was identical to the liquid level in the oil bath. The fermentation liquid was heated with stirring at 400±20 rpm using a magnetic stirrer (KF-82M) manufactured by YAZAWA Science Co., Ltd. As a result, the fermentation liquid samples 2 and 3 showed foam more than 7 cm high from the liquid level five minutes to ten minutes after the heating was started. Surprisingly, the fermentation liquid sample 1 remained without foam, and the same results as in the foamability test in Example 3 were verified.

### Reference Example 5 Measurement of Transmission of Ethanol Fermentation Liquid Sample

For measurement, an UV-Vis measurement device (V750) manufactured by JASCO Corporation was used as an ultraviolet and visible spectrophotometer, and 10 mm square quartz cuvettes manufactured by JASCO Corporation were used. Distilled water, 2 mL, manufactured by Wako Pure Chemical Industries, Ltd., was added to a cuvette, and the transmission background was measured by irradiation with beams having wavelengths of 200 nm to 800 nm. Thereafter, the fermentation liquid was added to an empty cuvette and subjected to measurement. The fermentation liquid was suitably diluted with distilled water to have a transmission of 0.5±0.1% T when irradiated with a beam having a wavelength of 300 nm. The transmission of the diluted liquid when irradiated with a beam having a wavelength of 600 nm was measured.

### Example 6 Determination of Threshold Value of Foamability of Ethanol Fermentation Liquid by Transmission

The fermentation liquid in an amount of 80 mL, prepared in the same manner as the sample 2 was added in an amount of 40 mL each, to 50 mL polypropylene conical tubes manufactured by Falcon, and centrifuged at 10,000 G for 60 minutes to separate particles and a supernatant. The precipitated particles were washed with water and centrifuged, three times each, and the supernatant was discarded after completion of the third centrifugation. The obtained particles were dried using a freeze-dryer (FDU-1200) manufactured by Tokyo Rikakikai Co., Ltd. The dry weight was 520 mg. These particles were diluted with water to prepare 104 mg/mL solutions (particle solutions). The prepared solutions were such that the volume ratio of the supernatant centrifuged at 10,000 G for 60 minutes to the particle solution was 1000:0, 997:3, 970:30, 900:100, 800:200, 700:300, and 600:400, respectively. In accordance with the transmission measurement method in Reference Example 5, the solutions were measured for transmission when irradiated with a beam having a wavelength of 600 nm and were found to show 97.6, 97.3, 91.8, 85.9, 84.5, 83.0, and 81.9, respectively. Each liquid was subjected to the distillation test in the same manner as in Example 5, and analyzed for transmission and foaming The obtained result was that, with a boundary at a transmission of 91% T based on a beam having a wavelength of 600 nm, no 7 cm or more foam was found from the liquid level at and above the transmission, and that some 7 cm or more foam was found at and below the transmission. Measurement of transmission is considered to be a method which makes it possible to measure foamability conveniently.

### Example 7 Measurement of Transmission of Fermentation Liquid Sample

In accordance with the transmission measurement method in Reference Example 5, the above-mentioned fermentation liquid sample 1, sample 2, and sample 3 were measured. As a result, the sample 1 showed a transmission of 94.7% T, the sample 2 showed 54.6% T, and the sample 3 showed 90.7% T, when irradiated with a beam having a wavelength of 600 nm. Thus, the results confirmed that, with a boundary at a transmission of 91% T of a beam having a wavelength of 600 nm, the actual fermentation liquid samples caused no foam above 91% T as in Example 3 and Reference Example 5, and, in contrast, caused foam at or below 91% T.

### INDUSTRIAL APPLICABILITY

An ethanol fermentation liquid obtained by the method of producing ethanol or an ethanol fermentation liquid can be utilized as fuel or industrial feedstock which is sustainable as what is called bio-ethanol.

1.-8. (canceled)

9. A method of producing ethanol, comprising:
a continuous ethanol fermentation step including:
culturing a microorganism with a fermentation feedstock containing cane molasses as a main component;
filtering the resulting culture liquid through a separation membrane to recover a filtrate containing said ethanol and from which said microorganism has been removed;
retaining or returning an unfiltered liquid containing said microorganism, in or to the culture liquid; and
adding an additional fermentation feedstock to said culture liquid; and
an ethanol concentration and purification step including distilling said filtrate which is collected in said continuous ethanol fermentation step and contains said ethanol;
wherein said microorganism causes a centrifugal supernatant of said culture liquid to contain particles having an average particle diameter of 100 nm or more, and
particles formed by the microorganism culture and contained in said filtrate containing ethanol have an average particle diameter of 40 to 80 nm.

10. The method of producing ethanol according to claim 9, wherein said particles have an average particle diameter of 300 nm or more.

11. The method of producing ethanol according to claim 9, wherein said microorganism is a yeast belonging to the genus Schizosaccharomyces.

12. The method of producing ethanol according to claim 9, wherein said particles formed by said microorganism culture and contained in said filtrate containing ethanol have a particle diameter distribution in a particle diameter range of 20 to 100 nm.

13. The method of producing ethanol according to claim 9, wherein the distillation is continuous distillation.

14. An ethanol fermentation liquid, comprising particles that are other than said microorganism produced by said microorganism culture and have an average particle diameter of 40 to 80 nm, wherein said ethanol fermentation liquid does not contain a component generated from hydrothermally-processed bagasse.

15. The ethanol fermentation liquid according to claim 14, wherein said particles have a particle diameter distribution in a particle diameter range of 20 to 100 nm.

16. An ethanol fermentation liquid showing a transmission of more than 91% T when irradiated with a beam having a wavelength of 600 nm, when said ethanol fermentation liquid is diluted with water to show a transmission of 0.5±0.1% T when irradiated with a beam having a wavelength of 300 nm.

17. The method of producing ethanol according to claim 10, wherein said microorganism is a yeast belonging to the genus Schizosaccharomyces.

18. The method of producing ethanol according to claim 10, wherein said particles formed by said microorganism culture and contained in said filtrate containing ethanol have a particle diameter distribution in a particle diameter range of 20 to 100 nm.

19. The method of producing ethanol according to claim 11, wherein said particles formed by said microorganism culture and contained in said filtrate containing ethanol have a particle diameter distribution in a particle diameter range of 20 to 100 nm.

20. The method of producing ethanol according to claim 10, wherein the distillation is continuous distillation.

21. The method of producing ethanol according to claim 11, wherein the distillation is continuous distillation.

22. The method of producing ethanol according to claim 12, wherein the distillation is continuous distillation.
